(19) United States
(12) Patent Application Publication
(10) Pub. No.: US 2026/0174804 A1
(43) Pub. Date: Jun. 25, 2026

(54) Amniotic Membrane Method of Use

(71) Applicant: Blake Smith OD, PA, Amarillo, TX (US)

(72) Inventor: Blake Smith, Amarillo, TX (US)

(21) Appl. No.: 19/182,905

(22) Filed: Apr. 18, 2025

Related U.S. Application Data
(60) Provisional application No. 63/738,144, filed on Dec. 23, 2024.

(51) Int. Cl. A61K 35/50; A61K 9/00

(52) U.S. Cl. CPC A61K 35/50; A61K 9/0051; A61L 27/3604

(57) ABSTRACT

A method for preoperative preparation to facilitate the therapeutic application of an amniotic membrane to an eye, leveraging its regenerative and anti-inflammatory properties to enhance corneal health prior to surgery. Comprising identifying patients requiring cataract surgery, diagnosing ocular surface conditions, applying the amniotic membrane to the eye to improve corneal health prior to biometric measurements, thereby ensuring enhanced accuracy in intraocular lens selection, allowing the therapeutic properties of the amniotic membrane to be absorbed by the eye, and evaluating the patient's ocular surface health after applying the amniotic membrane.

### CROSS-REFERENCE TO RELATED APPLICATIONS

This application claims benefit to US provisional patent application number 63/738,144 filed on 2024-12-23, which is hereby incorporated by reference.

### STATEMENT REGARDING FEDERALLY SPONSORED RESEARCH OR DEVELOPMENT (IF APPLICABLE)

Not applicable.

### REFERENCE TO SEQUENCE LISTING, A TABLE, OR A COMPUTER PROGRAM LISTING COMPACT DISC APPENDIX (IF APPLICABLE)

Not applicable.

### BACKGROUND OF THE INVENTION

Cataract surgery is one of the most common and successful surgical procedures performed worldwide, with millions of patients undergoing the procedure annually to restore vision compromised by lens opacification. Despite its widespread success, certain preoperative challenges persist, particularly in relation to the accuracy of biometric readings required for optimal intraocular lens (IOL) selection and placement. These challenges are exacerbated in patients with ocular surface disorders, such as dry eye syndrome or corneal irregularities, which can distort these measurements and lead to suboptimal surgical outcomes.

The Applicant has observed that patients undergoing cataract surgery often experience reduced quality of vision post-procedure due to inadequate preoperative preparation of the ocular surface. In cases of dryness or poor corneal health, the accuracy of biometric assessments is compromised, resulting in IOL selections based on flawed data. Consequently, even technically successful surgeries may fail to deliver optimal long-term visual improvement.

Beyond surgical preparation, dry eye syndrome affects millions globally, causing discomfort, visual disturbances, and potential corneal damage. Traditional treatments like artificial tears offer temporary relief but fail to address underlying causes, leaving a gap in effective, lasting solutions for both preoperative optimization and dry eye management.

Patients presenting for cataract surgery frequently exhibit additional ocular complications, such as chronic dry eye, corneal inflammation, or epithelial damage. Conventional preoperative strategies—including artificial tears, steroids, and topical therapies—often fall short of restoring the ocular surface to a healthy state, leading to inaccurate biometric measurements and poor-quality preoperative imaging. This increases the risk of suboptimal IOL calculation, selection, implantation, and alignment, as well as reduced post-surgical visual acuity. Similarly, severe corneal injuries from burns, infections, trauma, or prolonged contact lens wear further complicate these challenges. While prior art documents the use of amniotic membranes for therapeutic purposes—such as healing ulcerative keratitis, promoting epithelial regrowth, or reducing inflammation—their potential in preoperative optimization and dry eye treatment remains unexplored.

Recent research underscores the need for systematic ocular surface optimization. An article in Cataract & Refractive Surgery Today notes that “an unstable tear film or corneal pathology can interfere with preoperative measurements, leading to refractive surprises and patient dissatisfaction post-surgery” (Cataract & Refractive Surgery Today, July 2019). This dissatisfaction arises from both poor vision and physical discomfort. The article advocates a questionnaire-based approach to identify ocular surface diseases like dry eye before surgery, enhancing biometric accuracy and IOL selection. However, this method leans heavily on diagnostics and topical treatments, lacking a robust therapeutic intervention.

The Applicant has developed a novel method employing amniotic membranes for both preoperative preparation in cataract surgery and standalone treatment of dry eye symptoms. By restoring corneal health and optimizing the ocular surface before biometric readings, this approach improves IOL calculation accuracy and surgical outcomes. Simultaneously, it offers a superior alternative to artificial tears for dry eye, leveraging the membranes'regenerative and anti-inflammatory properties to promote healing and address root causes rather than merely alleviating symptoms. Unlike prior art's diagnostic focus, this invention provides a tangible, therapeutic solution, enhancing preoperative diagnostics, postoperative vision stability, and overall ocular health.

Moreover, the application of amniotic membranes holds significant potential in the field of laser refractive surgery. By optimizing the ocular surface preoperatively, the method can ensure more accurate corneal mapping and laser ablation, leading to improved surgical precision and patient outcomes. Additionally, post-operatively, the regenerative properties of the membrane can accelerate corneal healing, reduce inflammation, and minimize the risk of complications such as haze or regression. This dual application—both before and after laser refractive surgery—positions the method as a versatile tool in enhancing the overall success of refractive surgeries.

This disclosure marks a significant advancement in optometry and ophthalmology, addressing critical gaps in current standards of care for cataract surgery preparation and dry eye treatment. By integrating amniotic membranes into these contexts, the method delivers benefits to patients and clinicians alike, overcoming the unpredictability of postoperative vision and the limitations of artificial tears.

No prior art known to the Applicant fully anticipates this dual-purpose application.

### BRIEF SUMMARY OF THE INVENTION

A method for preoperative preparation 200 to facilitate the therapeutic application of an amniotic membrane 102 to an eye 104, leveraging its regenerative and anti-inflammatory properties to enhance corneal health prior to surgery. Comprising identifying patients requiring cataract surgery, diagnosing ocular surface conditions, applying said amniotic membrane 102 to said eye 104 to improve corneal health prior to biometric measurements, thereby ensuring enhanced accuracy in intraocular lens selection, allowing the therapeutic properties of said amniotic membrane 102 to be absorbed by said eye 104, and evaluating the patient's ocular surface health after applying said amniotic membrane 102.

A method of ocular surface optimization using the amniotic membrane 102 in combination with a contact lens 106. Comprising preparing said amniotic membrane 102 by ensuring its sterility, placing said amniotic membrane 102 inside said contact lens 106 prior to application on the eye 104, positioning said contact lens 106 and said amniotic membrane 102 onto said eye 104 such that said amniotic membrane 102 contacts the cornea, maintaining said contact lens 106 in place for a time sufficient to allow regenerative and anti-inflammatory factors to absorb into said eye 104, and removing said contact lens 106 or said amniotic membrane 102 after verifying an improvement in corneal health or reduction in dry eye symptoms.

The method for preoperative preparation 200 to facilitate the therapeutic application of the amniotic membrane 102 to the eye 104, leveraging its regenerative and anti-inflammatory properties to enhance corneal health prior to surgery. Comprising identifying patients requiring cataract surgery. diagnosing ocular surface conditions. Applying said amniotic membrane 102 to said eye 104 to improve corneal health prior to biometric measurements, thereby ensuring enhanced accuracy in intraocular lens selection. Allowing the therapeutic properties of said amniotic membrane 102 to be absorbed by said eye 104. evaluating the patient's ocular surface health after applying said amniotic membrane 102. preparing said amniotic membrane 102 by ensuring its sterility and sizing it for application. placing said amniotic membrane 102 onto the ocular surface beneath the contact lens 106 to secure it in position. repeating the application of said amniotic membrane 102 if a corneal assessment 504 indicates incomplete healing.

### BRIEF DESCRIPTION OF THE SEVERAL VIEWS OF THE DRAWING

FIG. 1 illustrates an ocular preparation system 100 comprising an amniotic membrane 102, an eye 104, and a contact lens 106.

FIG. 2A illustrates a flowchart of a method for preoperative preparation 200 using said ocular preparation system 100.

FIG. 2B illustrates a flowchart of a method for treating dry eye symptoms 212 using said ocular preparation system 100.

FIG. 3 illustrates a flowchart detailing a diagnosis and evaluation phase 300.

FIG. 4A illustrates a flowchart of an amniotic membrane application procedure 400.

FIG. 4B illustrates an alternative flowchart of an amniotic membrane application procedure 410.

FIG. 5 illustrates a flowchart for a follow-up and evaluation process 500.

FIG. 6 illustrates a flowchart demonstrating a pre-surgical biometric optimization phase 600.

FIG. 7 illustrates a flowchart of an alternative embodiment 700 for managing severe ocular surface injuries prior to cataract surgery.

FIG. 8 illustrates a flowchart of a method for preoperative preparation prior to laser refractive surgery 800 using the ocular preparation system 100.

FIG. 9 illustrates a flowchart of a method for post-laser refractive surgery corneal healing 900 using the ocular preparation system 100.

### DETAILED DESCRIPTION OF THE INVENTION

The following description is presented to enable any person skilled in the art to make and use the invention as claimed and is provided in the context of the particular examples discussed below, variations of which will be readily apparent to those skilled in the art. In the interest of clarity, not all features of an actual implementation are described in this specification. It will be appreciated that in the development of any such actual implementation (as in any development project), design decisions must be made to achieve the designers'specific goals (e.g., compliance with system-and business-related constraints), and that these goals will vary from one implementation to another. It will also be appreciated that such development effort might be complex and time-consuming, but would nevertheless be a routine undertaking for those of ordinary skill in the field of the appropriate art having the benefit of this disclosure. Accordingly, the claims appended hereto are not intended to be limited by the disclosed embodiments, but are to be accorded their widest scope consistent with the principles and features disclosed herein.

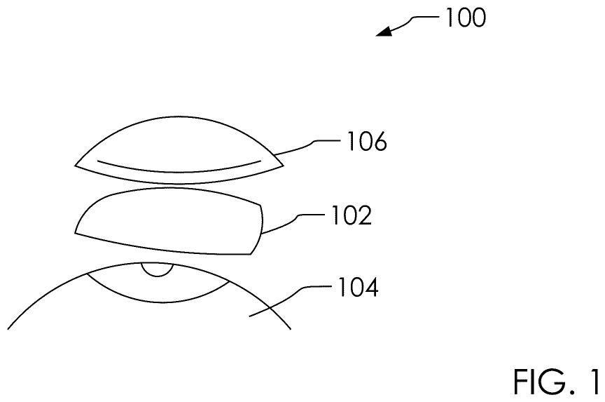

FIG. 1 illustrates an ocular preparation system 100 comprising an amniotic membrane 102, an eye 104, and a contact lens 106.

In one embodiment, said amniotic membrane 102 is positioned over said eye 104, with said contact lens 106 placed over it to ensure correct positioning and provide certainty of its location on the ocular surface. This configuration leverages the regenerative and anti-inflammatory properties of said amniotic membrane 102 to enhance corneal health, either for preoperative optimization or dry eye treatment. In another embodiment, said amniotic membrane 102 is placed inside said contact lens 106 before application to said eye 104, simplifying the process and ensuring stability. Alternatively, said amniotic membrane 102 can be applied directly to said eye 104 without said contact lens 106, allowing sufficient time for its healing properties to absorb. A hybrid contact lens membrane, combining both components, is contemplated as a manufacturer-developed enhancement.

In some embodiments, application may occur with or without said contact lens 106, depending on clinical needs.

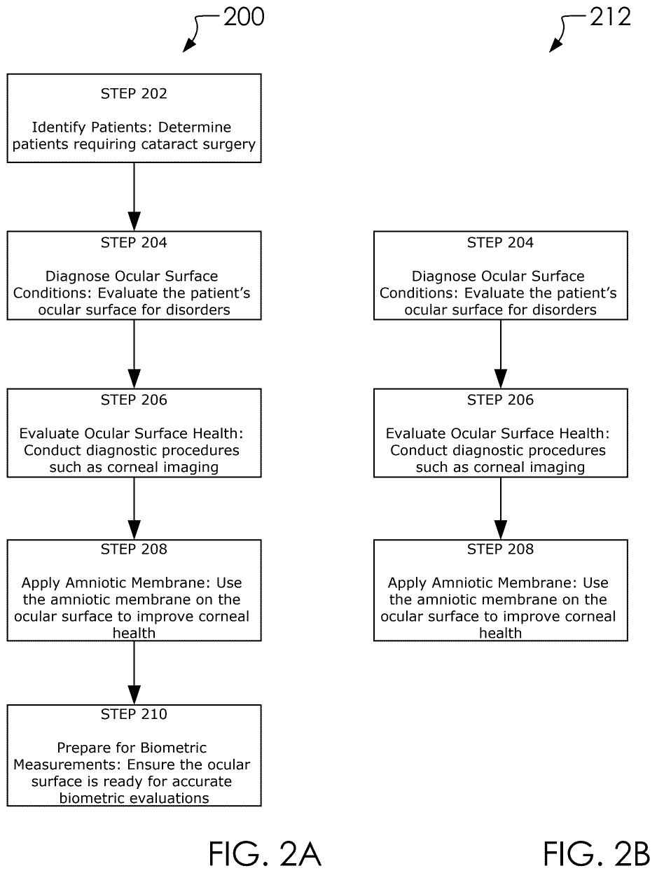

FIG. 2A illustrates a flowchart of a method for preoperative preparation 200 using said ocular preparation system 100.

Said method for preoperative preparation 200 begins by identifying patients requiring cataract surgery (STEP 202), followed by diagnosing ocular surface conditions (STEP 204). The ocular surface health is evaluated via diagnostic procedures like corneal imaging (STEP 206). Subsequently, said amniotic membrane 102 is applied to improve corneal health (STEP 208), preparing the surface for accurate biometric measurements (STEP 210).

In all cases, an anesthetic can be provided to a patient to ensure comfort during the application of said amniotic membrane 102 and/or said contact lens 106.

FIG. 2B illustrates a flowchart of a method for treating dry eye symptoms 212 using said ocular preparation system 100.

Said method for treating dry eye symptoms 212 starts with diagnosing ocular surface conditions (STEP 252), followed by evaluating ocular surface health through diagnostics such as corneal imaging (STEP 254). Said amniotic membrane 102 is then applied to alleviate dry eye symptoms and enhance corneal health (STEP 256), offering a lasting alternative to artificial tears.

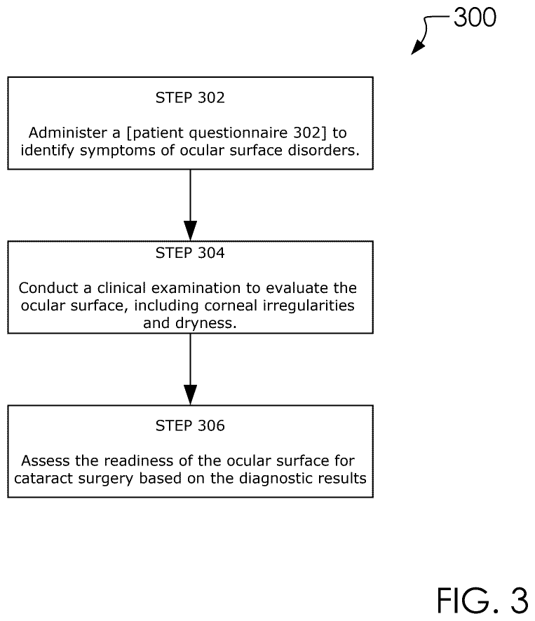

FIG. 3 illustrates a flowchart detailing a diagnosis and evaluation phase 300.

Said diagnosis and evaluation phase 300 includes administering a patient questionnaire 302 to identify ocular surface disorder symptoms (STEP 302) and conducting a clinical examination 304 to assess corneal irregularities or dryness (STEP 304). The readiness of the ocular surface for surgery or the severity of dry eye is then determined (STEP 306).

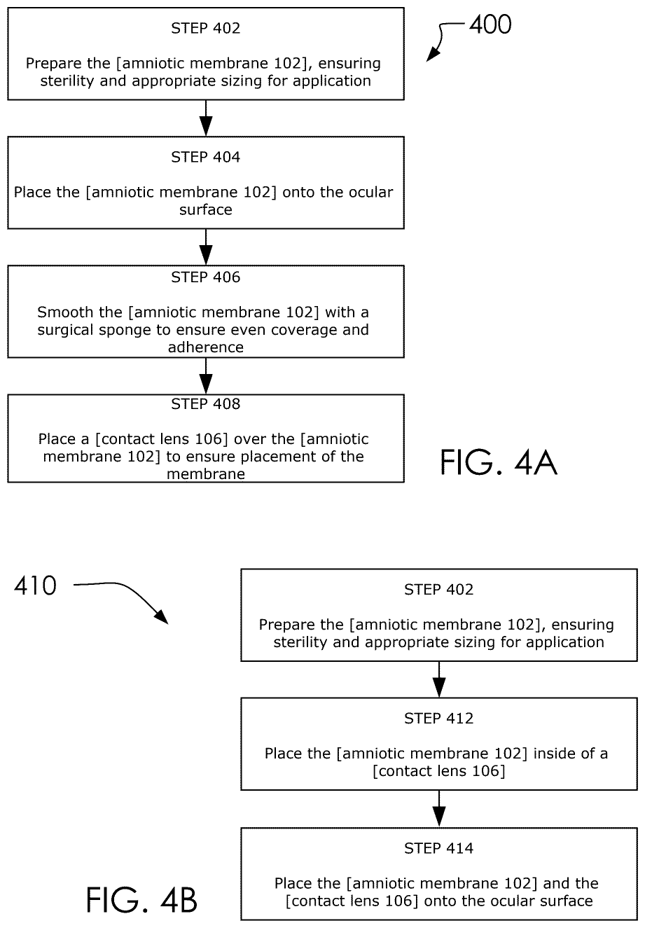

FIG. 4A illustrates a flowchart of an amniotic membrane application procedure 400.

Said amniotic membrane application procedure 400 begins by preparing said amniotic membrane 102, ensuring sterility and proper sizing (STEP 402). Said amniotic membrane 102 is placed onto the ocular surface (STEP 404), smoothed with a surgical sponge for even coverage and adherence (STEP 406), and secured with said contact lens 106 to confirm its position (STEP 408).

FIG. 4B illustrates an alternative flowchart of an amniotic membrane application procedure 410.

In addition to its use in cataract surgery preparation and dry eye treatment, the amniotic membrane application procedures described herein can be adapted for laser refractive surgery. For preoperative preparation, said amniotic membrane 102 can be applied to optimize the ocular surface, ensuring accurate corneal measurements for laser ablation planning. Post-operatively, said amniotic membrane 102 can be applied to promote rapid healing and reduce inflammation, enhancing patient recovery. These adaptations utilize the same regenerative and anti-inflammatory properties of said amniotic membrane 102, tailored to the specific needs of refractive surgery.

In this embodiment, said amniotic membrane application procedure 410 starts with preparing said amniotic membrane 102 (STEP 412), placing it inside said contact lens 106 (STEP 414), and applying the combined unit to the ocular surface (STEP 416), enhancing procedural efficiency.

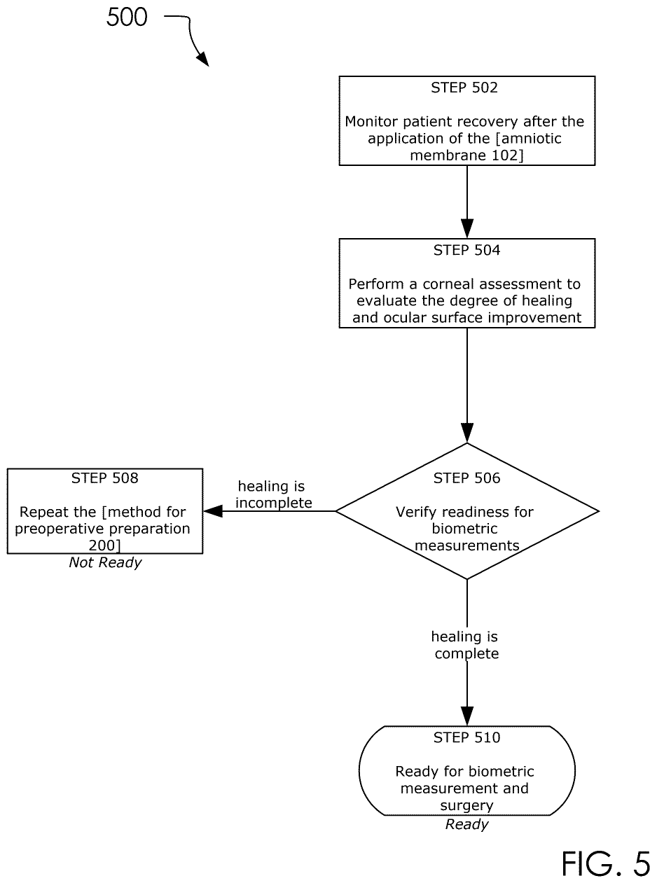

FIG. 5 illustrates a flowchart for a follow-up and evaluation process 500.

Said follow-up and evaluation process 500 monitors patient recovery post-application of said amniotic membrane 102 (STEP 502). A corneal assessment 504 evaluates healing and ocular surface improvement (STEP 504), with photographic evidence (e.g., via microscope or camera) confirming membrane centering and documenting progress (STEP 506). If the surface is ready for biometric measurements or dry eye symptoms are sufficiently improved, the process advances (STEP 510); otherwise, said method for preoperative preparation 200 or method for treating dry eye symptoms 212 may be repeated (STEP 508).

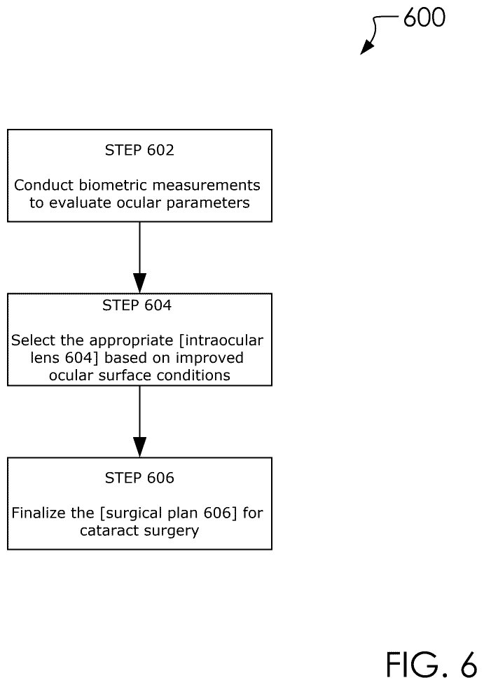

FIG. 6 illustrates a flowchart demonstrating a pre-surgical biometric optimization phase 600.

Said pre-surgical biometric optimization phase 600 involves conducting biometric measurements 602 to assess ocular parameters (STEP 602), selecting an intraocular lens 604 based on the optimized ocular surface (STEP 604), and finalizing a surgical plan 606 for cataract surgery (STEP 606).

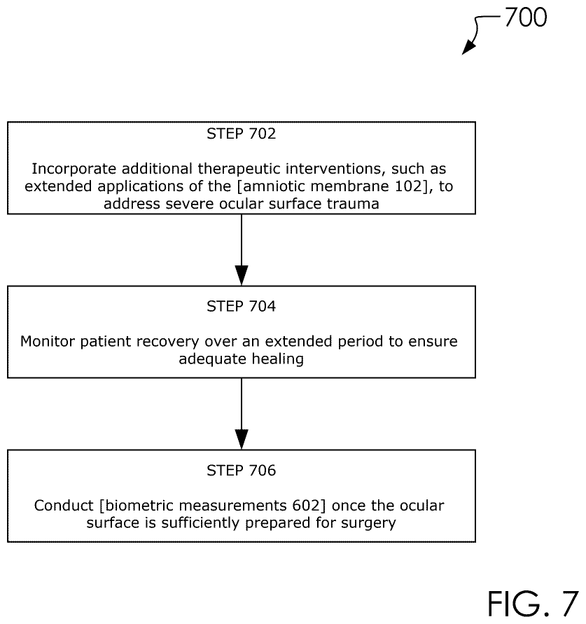

FIG. 7 illustrates a flowchart of an alternative embodiment 700 for managing severe ocular surface injuries prior to cataract surgery.

Said alternative embodiment 700 employs extended applications of said amniotic membrane 102 to treat severe trauma (STEP 702), monitors recovery over time (STEP 704), and conducts biometric measurements 602 once the surface is adequately healed (STEP 706).

These methods harness the regenerative and anti-inflammatory properties of said amniotic membrane 102 to optimize ocular surface health, improve preoperative diagnostics, and treat dry eye symptoms effectively. In practice, associated Current Procedural Terminology (CPT) codes—65778 (placement of amniotic membrane), 92071 (contact lens fitting for ocular surface disease), and 92285 (external ocular photography)—standardize billing and reimbursement, ensuring practitioners can implement these procedures with known inputs and outcomes.

Additional Considerations

In some procedures, eyelid retraction using a speculum is beneficial if the patient's involuntary blinking impedes accurate membrane placement. It is likewise contemplated that in certain cases no contact lens is used at all; so long as said amniotic membrane 102 remains on said eye 104 for a sufficient duration to deliver its therapeutic properties, the underlying principles remain the same.

Practitioners may also wish to code these procedures under recognized insurance or billing classifications, such as CPT codes 65778, 92071, and 92285. By having discrete, recognized procedure codes, doctors can streamline the integration of amniotic membrane therapy —whether for preoperative conditioning or the alleviation of dry eye—into their existing treatment protocols.

Throughout this disclosure, the term “amniotic membrane” is intended to encompass both cryopreserved and dehydrated amniotic tissues, as well as potential future variations, including hybrid contact-lens-and-membrane assemblies designed by manufacturers. Monitoring steps can include photographic or microscopic evidence to confirm that said amniotic membrane 102 remains centered on said eye 104, thereby providing verifiable data on healing and corneal stability over time.

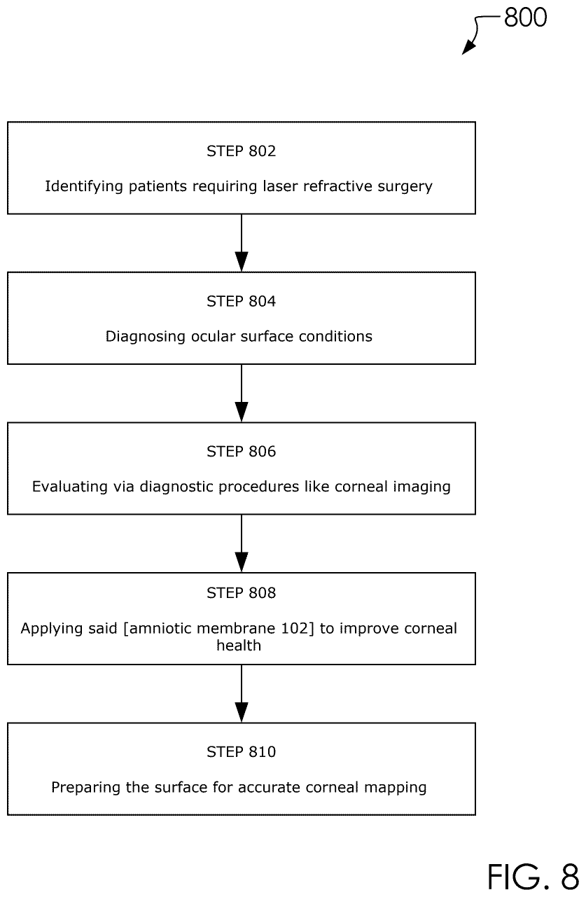

FIG. 8 illustrates a flowchart of a method for preoperative preparation prior to laser refractive surgery 800 using the ocular preparation system 100.

Said method for preoperative preparation prior to laser refractive surgery 800 begins by identifying patients requiring laser refractive surgery (STEP 802), followed by diagnosing ocular surface conditions (STEP 804). The ocular surface health is evaluated via diagnostic procedures like corneal imaging (STEP 806). Subsequently, said amniotic membrane 102 is applied to improve corneal health (STEP 808), preparing the surface for accurate corneal mapping (STEP 810).

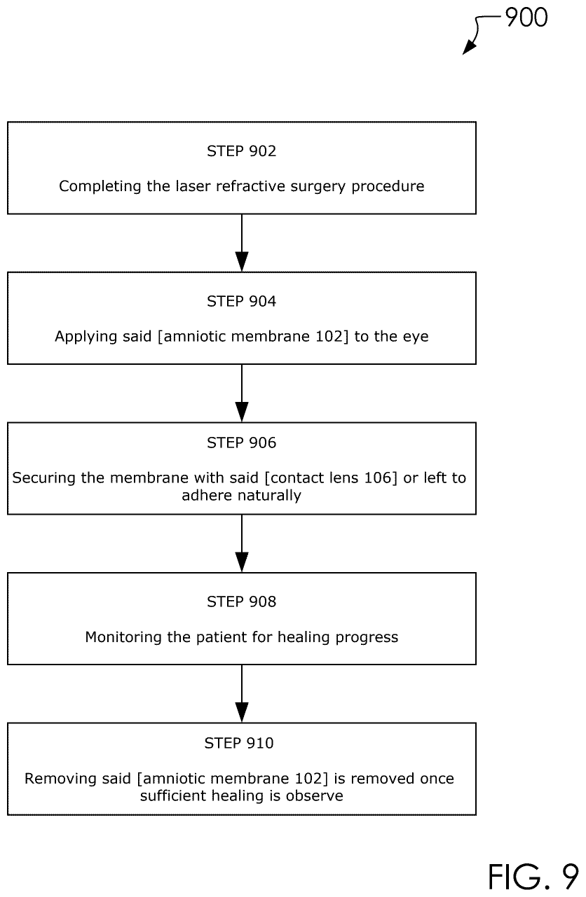

FIG. 9 illustrates a flowchart of a method for post-laser refractive surgery corneal healing 900 using the ocular preparation system 100.

Said method for post-laser refractive surgery corneal healing 900 begins by completing the laser refractive surgery procedure (STEP 902), followed by applying said amniotic membrane 102 to the eye (STEP 904). The membrane is then secured with said contact lens 106 or left to adhere naturally (STEP 906). The patient is monitored for healing progress (STEP 908).

### PARTS LIST

the ocular preparation system 100,

the amniotic membrane 102,

the eye 104,

the contact lens 106,

the method for preoperative preparation 200,

the method for treating dry eye symptoms 212,

the diagnosis and evaluation phase 300,

the patient questionnaire 302,

the clinical examination 304,

the amniotic membrane application procedure 400,

the amniotic membrane application procedure 410,

the follow-up and evaluation process 500,

the corneal assessment 504,

the pre-surgical biometric optimization phase 600,

the biometric measurements 602,

the intraocular lens 604,

the surgical plan 606,

the alternative embodiment 700,

the method for preoperative preparation prior to laser refractive surgery 800, and

the method for post-laser refractive surgery corneal healing 900.

The following section comprises one preferred embodiment of the disclosure and is based on the original claims of this application.

The method for preoperative preparation 200 to facilitate the therapeutic application of the amniotic membrane 102 to the eye 104, leveraging its regenerative and anti-inflammatory properties to enhance corneal health prior to surgery. Can comprise identifying patients requiring cataract surgery, diagnosing ocular surface conditions, applying said amniotic membrane 102 to said eye 104 to improve corneal health prior to biometric measurements, thereby ensuring enhanced accuracy in intraocular lens selection, allowing the therapeutic properties of said amniotic membrane 102 to be absorbed by said eye 104, and evaluating the patient's ocular surface health after applying said amniotic membrane 102.

Preparing said amniotic membrane 102 by ensuring its sterility and sizing it for application, and placing said amniotic membrane 102 onto the ocular surface beneath the contact lens 106 to secure it in position.

Smoothing said amniotic membrane 102 with a surgical sponge during application to ensure even coverage and adherence.

Monitoring the patient for initial discomfort and ensuring correct placement of said contact lens 106.

Monitoring patient recovery after applying said amniotic membrane 102, and conducting the corneal assessment 504 to evaluate healing and ocular surface improvement.

Repeating the application of said amniotic membrane 102 if said corneal assessment 504 indicates incomplete healing.

Conducting biometric measurements 602 to assess ocular parameters, selecting the intraocular lens 604 based on the optimized ocular surface, and finalizing the surgical plan 606 for cataract surgery.

Incorporating extended applications of said amniotic membrane 102 to address significant corneal trauma, and monitoring recovery before conducting biometric measurements 602.

A method of ocular surface optimization using the amniotic membrane 102 in combination with the contact lens 106, the method can comprise preparing said amniotic membrane 102 by ensuring its sterility, placing said amniotic membrane 102 inside said contact lens 106 prior to application on the eye 104, positioning said contact lens 106 and said amniotic membrane 102 onto said eye 104 such that said amniotic membrane 102 contacts the cornea, maintaining said contact lens 106 in place for a time sufficient to allow regenerative and anti-inflammatory factors to absorb into said eye 104, and removing said contact lens 106 or said amniotic membrane 102 after verifying an improvement in corneal health or reduction in dry eye symptoms.

Smoothing said amniotic membrane 102 with a surgical sponge prior to placing said contact lens 106 and said amniotic membrane 102 onto said eye 104, ensuring even contact with the corneal surface.

Capturing microscopic or photographic documentation to confirm that said amniotic membrane 102 remains centered on said eye 104 during the duration of said application.

Said amniotic membrane 102 comprises a cryopreserved or dehydrated membrane having regenerative and anti-inflammatory properties effective for either preoperative preparation or chronic dry eye relief.

Using said amniotic membrane 102 for both preoperative ocular surface optimization and standalone dry eye treatment, wherein the same placement procedure can be adaptable based on the patient's surgical or non-surgical needs.

The method for preoperative preparation 200 to facilitate the therapeutic application of the amniotic membrane 102 to the eye 104, leveraging its regenerative and anti-inflammatory properties to enhance corneal health prior to surgery. Can comprise identifying patients requiring cataract surgery. Diagnosing ocular surface conditions. Applying said amniotic membrane 102 to said eye 104 to improve corneal health prior to biometric measurements, thereby ensuring enhanced accuracy in intraocular lens selection. Allowing the therapeutic properties of said amniotic membrane 102 to be absorbed by said eye 104. Evaluating the patient's ocular surface health after applying said amniotic membrane 102. Preparing said amniotic membrane 102 by ensuring its sterility and sizing it for application. Placing said amniotic membrane 102 onto the ocular surface beneath the contact lens 106 to secure it in position. Repeating the application of said amniotic membrane 102 if the corneal assessment 504 indicates incomplete healing.

Smoothing said amniotic membrane 102 with a surgical sponge during application to ensure even coverage and adherence.

Monitoring the patient for initial discomfort and ensuring correct placement of said contact lens 106.

Monitoring patient recovery after applying said amniotic membrane 102, and conducting the corneal assessment 504 to evaluate healing and ocular surface improvement.

Conducting biometric measurements 602 to assess ocular parameters, selecting the intraocular lens 604 based on the optimized ocular surface, and finalizing the surgical plan 606 for cataract surgery.

Incorporating extended applications of said amniotic membrane 102 to address significant corneal trauma, and monitoring recovery before conducting biometric measurements 602.

Various changes in the details of the illustrated operational methods are possible without departing from the scope of the following claims. Some embodiments may combine the activities described herein as being separate steps. Similarly, one or more of the described steps may be omitted, depending upon the specific operational environment the method is being implemented in. It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

1. A method for improving corneal health prior to cataract surgery in a patient; the method consisting of:
identifying a patient in need of cataract surgery,
diagnosing an ocular surface condition in the patient that requires therapeutic treatment,
preparing a dehydrated amniotic membrane by verifying that it is sterile and properly sized for the patient's eye,
applying said dehydrated amniotic membrane to said eye measured visual acuity of the subject following intraocular lens placement,
allowing regenerative and anti-inflammatory factors of said amniotic membrane to be absorbed by said eye, and
evaluating the ocular surface health of the patient after said applying step.

2. The method for improving corneal health prior to cataract surgery in a patient of claim 1, wherein applying said amniotic membrane to said eye comprises:
preparing said amniotic membrane by ensuring its sterility and sizing it for application, and p1 placing said amniotic membrane onto the ocular surface beneath a contact lens to secure it in position.

3. The method for improving corneal health prior to cataract surgery in a patient of claim 2, further comprising:
smoothing said amniotic membrane with a surgical sponge during application to ensure even coverage and adherence.

4. The method for improving corneal health prior to cataract surgery in a patient of claim 2, further comprising:
monitoring for initial discomfort after said applying step and ensuring that said amniotic membrane remains correctly positioned on the eye.

5. The method for improving corneal health prior to cataract surgery in a patient of claim 1, further comprising:
monitoring the healing of the corneal epithelium after applying said amniotic membrane, and
conducting a corneal assessment to evaluate healing and ocular surface improvement.

6. The method for improving corneal health prior to cataract surgery in a patient of claim 5, further comprising:
repeating the application of said amniotic membrane if said corneal assessment indicates that the corneal epithelium has not fully healed.

7. The method for improving corneal health prior to cataract surgery in a patient of claim 1, further comprising:
performing biometric measurements on the patient's eye after the corneal health is improved,
selecting an intraocular lens for the cataract surgery based on the improved ocular surface condition.

8. The method for improving corneal health prior to cataract surgery in a patient of claim 1, further comprising:
maintaining said amniotic membrane on the eye for at least one week as an extended application to address corneal trauma, and
monitoring the patient's recovery prior to biometric measurement.

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. A method for preparing a patient for cataract surgery, the method consisting of:
identifying a patient scheduled for cataract surgery;
diagnosing dry eye or corneal surface irregularity in the patient;
placing a sterile, dehydrated amniotic membrane directly onto the ocular surface of the patient's eye;
covering the dehydrated amniotic membrane with a bandage contact lens to secure the membrane in position;
maintaining the dehydrated amniotic membrane on the eye for a period sufficient to permit therapeutic absorption; and
thereafter performing biometric measurements for intraocular lens selection after improvement of the ocular surface.

33. The method of claim 32, wherein therapeutic absorption is substantially complete prior to removal of the bandage contact lens.
